(19) United States
(12) Patent Application Publication
(10) Pub. No.: US 2017/0189036 A1
(43) Pub. Date: Jul. 6, 2017

(54) Drilling Assembly and a Method for Preparing Graft Socket for Ligament Reconstruction

(71) Applicant: PATWARDHAN APOORVA RAJEEV, PUNE (IN)

(72) Inventor: PATWARDHAN APOORVA RAJEEV, PUNE (IN)

(21) Appl. No.: 14/983,581

(22) Filed: Dec. 30, 2015

(51) Int. Cl. A61B 17/16; A61B 90/00; A61B 17/32; A61B 1/317; A61B 17/88

(52) U.S. Cl. CPC A61B 17/1635; A61B 1/317; A61B 17/1659; A61B 17/1675; A61B 17/1628; A61B 17/1622; A61B 17/8897; A61B 17/320016; A61B 90/06; A61B 90/03; A61B 17/1633; A61B 2090/061; A61B 2090/034

(57) ABSTRACT

Disclosed is a drilling assembly for anatomic anterior cruciate ligament reconstruction and a method therefore. The drilling assembly comprises an endoscopic reamer, a variable position external stopper and a fixed length outer protective sleeve. The endoscopic reamer has dual length markings thereon. The drilling assembly and the method make the tunnel reaming precise and independent of arthroscopic visualization and manual judgments by avoiding over drilling as well as protect the cartilage over the medial femoral condyle and fibers of Posterior Cruciate Ligament during introduction of the femoral endoscopic reamer over the guide wire during anatomic anterior cruciate ligament reconstruction.

### FIELD OF THE INVENTION

The present invention relates generally to an assembly and method for graft socket preparation during ligament reconstruction and more particularly, to a drilling assembly and a method for graft socket preparation during anatomicanterior cruciate ligament reconstruction configured to stop the drilling at a predefined length making the process independent of operator judgement thereby reducing humanly errors.

### BACKGROUND OF THE INVENTION

For any ligament reconstruction involving cortical suspension devices, following steps are followed. For the purpose of explanation only an anatomic Anterior Cruciate Ligament (ACL) reconstruction is described and illustrated in FIGS. 1 to 4. Further, a femoral tunnel preparation is done with the knee flexed at 120 degrees or higher. First, a 2.4 mm guide wire is passed and drilled through an Antero Medial (AM) portal at an anatomic ACL insertion site.

Thereafter, a 4.5 or 5 mm drill is passed over the guide wire and a tunnel is drilled in a lateral femoral condyle. Following this, both the drill and the guide wire are removed. The length of the tunnel is measured using a depth gauge.

The surgeons subtract about 5-10 mm from the total tunnel length and drill for the ACL femoral graft socket tunnel using the endoscopic reamer that corresponds to the diameter of the ACL graft, taking care not to burst the lateral cortex. When using the femoral cortical suspension system, lateral cortex burst leaves no cortex intact for the metal part of the femoral cortical suspension system to be seated horizontally.

### DISADVANTAGES OF THE EXISTING SYSTEM

Hyperflexion of the knee results in a loss of joint distention due to external compression of the knee joint capsule by the soft tissue of the thigh. This can result in bleeding and a loss of joint visualization due to encroachment of the fat pad into the notch. Hyperflexion of the knee and bone debris produced during femoral ACL graft socket tunnel preparation restricts arthroscopic vision making the reaming tricky and operator dependent and thus prone to manual error or wrong judgment. If the fat pad obscures the vision, it is resected using arthroscopic resection system. In order to maintain adequate joint distention and visualization in hyperflexed position, the fluid pressure needs to be increased while working in the notch and motorized shaver blade needs to be inserted through an additional portal to suck bone debris and clear the fat pad.

Poor visualization due to hyperflexed knee joint, and bone debris generated during femoral graft socket tunnel drilling warrants slow and careful drilling of the femoral ACL graft socket tunnel. The drill bit markings, in mm; over the femoral endoscopic drill is the only way to judge the depth of the drilling. Drilling the femoral ACL graft socket tunnel close to the lateral cortex allows easy flipping of the femoral suspension system. But error in judgment and poor visualization can cause over drilling of the femoral ACL graft socket leading to femoral cortical burst which means there is no cortex left intact to support the horizontal seating of the metal component of femoral suspension system. Thus, there is no protective mechanism in the current technique, to avoid lateral cortex perforation with the endoscopic reamer.

Advancement of the reamer over the guide wire through the AM portal can cause damage to the cartilage over the Medial Femoral Condyle (MFC) and fibers of the Posterior Cruciate Ligament (PCL). Passing pin maneuver and angling away of guide wire are the existing ways to avoid damage to these structures during drilling.

Therefore, there is a need to provide a drilling assembly for anatomic anterior cruciate ligament reconstruction and a method that overcomes the above-mentioned drawbacks of the prior art.

### OBJECTS OF THE INVENTION

An object of the present invention is to make a ligament graft socket tunnel reaming precise and independent of arthroscopic visualization and manual judgments.

Another object of the present invention is to protect the cartilage over the Medial Femoral Condyle (MFC) and fibers of Posterior Cruciate Ligament (PCL) during introduction of a femoral endoscopic reamer over the guide wire specifically for anatomic anterior cruciate ligament reconstruction.

### SUMMARY OF THE INVENTION

Accordingly, the present invention, in an aspect provides a drilling assembly for ligament reconstruction. In the present embodiment, the reconstruction of an Anterior Cruciate Ligament (ACL) is explained. The ACL reconstruction is carried out by passing and drilling a guide wire through a femoral aimer with knee flexed at 120 degrees through an Antero Medial (AM) portal at an anatomical ACL femoral insertion site for determining the direction of a femoral ACL graft socket tunnel Thereafter a larger drill of 4.5 or 5 mm diameter is passed over the guide wire to drill a tunnel in the lateral femoral condyle. The length of the tunnel is measured using a depth gauge. The length of the ACL graft socket is decided by the surgeon based on the measured tunnel length. The drilling assembly comprises an endoscopic reamer, an outer sleeve and a stopper.

The endoscopic reamer is a femoral endoscopic drill of a size ranging between 6 mm to 11 mm. The size of the endoscopic reamer is selected based on the size of the graft to be inserted at the ACL insertion site. The endoscopic reamer includes at least one measurement marking thereon. Typically, the endoscopic reamer has dual measurement markings indicated thereon. The endoscopic reamer is adapted to slidably pass over the guide wire for drilling a predefined length marking to prepare the femoral ACL graft socket tunnel in the lateral femoral condyle for the insertion of an anterior cruciate ligament graft therein.

The outer sleeve is a fixed length outer protective sleeve of slightly wider diameter than the selected endoscopic reamer adapted to be slidably passed over the guide wire and advanced over the guide wire to rest on the medial wall of the lateral femoral condyle to protect the cartilage over the Medial Femoral Condyle (MFC) and fibers of Posterior Cruciate Ligament (PCL) during drilling. The outer sleeve includes a handle configured thereon to provide grip to the surgeon. The cylindrical part of the outer sleeve resting over the medial wall of the lateral femoral condyle can be either full circumference or half circumference throughout the entire length up to the handle. The outer sleeve with full circumference cylindrical portion includes a window configured to allow the surgeon to arthroscopically confirm the drilling of the femoral ACL graft socket tunnel upto the predefined length. The window is configured at an end portion of the outer sleeve.

The cylindrical part of the outer sleeve with half circumference throughout the length makes insertion through the AM portal easier providing more visibility to the reamer thereby obviating the need of a window.

The stopper adapted to be preloaded and secured to the endoscopic reamer at a portion on the endoscopic reamer enabling the stopping of the travel of the endoscopic reamer for drilling hole of predefined length of the femoral ACL graft socket tunnel The endoscopic reamer along with the stopper is adapted to be attached on a motorized drill for passing over the guide wire and moving inside the outer sleeve, drilling a tunnel in the lateral femoral condyle until the stopper hits the outer sleeve thereby avoiding over drilling of the femoral ACL graft socket tunnel beyond the predefined length. The stopper is a variable external stopper having a diameter wider than that of the outer sleeve so as to hit the outer sleeve upon reaching at least one measurement marking on the endoscopic reamer corresponding to the predefined length of the femoral ACL graft socket tunnel to be drilled thereby avoiding over drilling.

In another aspect, the present invention provides a method for preparing graft socket tunnel during anatomic anterior cruciate ligament reconstruction.

### BRIEF DESCRIPTION OF FIGURES

The advantages and features of the present invention will be better understood with reference to the following detailed description and claims taken in conjunction with the accompanying drawings, wherein like elements are identified with like symbols, and in which:

FIG. 1 shows schematic view of a guide wire passed in the bone at the anatomical ACL insertion site through the AM portal, in accordance with a prior art;

FIG. 2 illustrates schematic view of an over drilling by using a drill of about 4.5 or 5 mm diameter, in accordance with the prior art;

FIG. 3 schematic view of depicting a depth gauge used for measuring the tunnel length in accordance with the prior art;

FIG. 4 illustrates a schematic view of reinsertion of a guide wire through the AM portal through the predrilled tunnel;

FIG. 5 illustrates schematic view of an outer sleeve of slightly wider diameter than the selected endoscopic reamer with full circumference cylindrical portion and window at the tip passing over the guide wire, in accordance with the present invention;

FIG. 5a shows an embodiment of the outer sleeve with half circumference cylindrical portion of FIG. 5;

FIG. 6 shows schematic view of a stopper fixed at 90+25=115 mm on an endoscopic reamer, in accordance with the present invention;

FIG. 6a shows an embodiment of the endoscopic reamer with dual measurement scale;

FIG. 7 illustrates a schematic view of a stopper that hits the outer sleeve and ensures about 25 mm tunnel during drilling, in accordance with the present invention;

FIG. 8 illustrates a schematic view of femoral ACL graft socket with intact lateral cortex capable of seating a horizontal femoral suspension device, in accordance with the present invention, and

FIG. 9 illustrates flow chart of the method of preparing the femoral ACL graft socket tunnel using the drilling assembly in accordance with the present invention.

### DETAILED DESCRIPTION OF THE INVENTION

For a thorough understanding of the present invention, reference is to be made to the following detailed description in connection with the above-described drawings. Although the present invention is described in connection with exemplary embodiments, the present invention is not intended to be limited to the specific forms set forth herein. It is understood that various omissions and substitutions of equivalents are contemplated as circumstances may suggest or render expedient. Also, it is to be understood that the phraseology and terminology used herein is for the purpose of description and should not be regarded as limiting.

The present invention provides a drilling assembly for preparing graft socket for an anatomic ligament reconstruction and a method thereof. The drilling assembly and method for a femoral ACL graft socket drilling for anatomic Anterior Cruciate Ligament (ACL) reconstruction is described in details. The drilling assembly and the method makes the femoral ACL graft socket tunnel reaming precise and independent of arthroscopic visualization and manual judgment as well as protects the cartilage of Medial Femoral Condyle (MFC) and fibers of Posterior Cruciate Ligament (PCL) during introduction of a femoral endoscopic reamer.

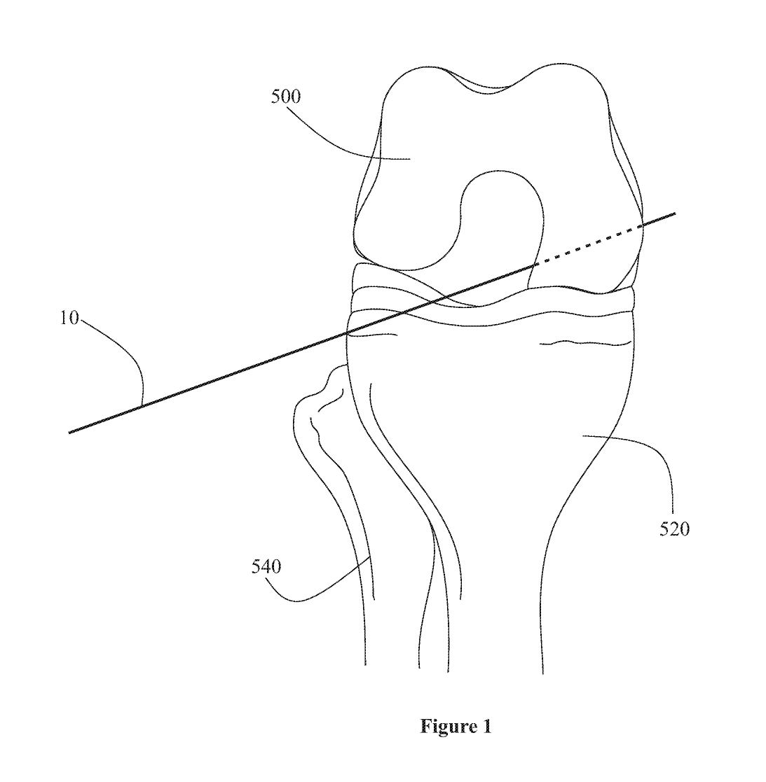

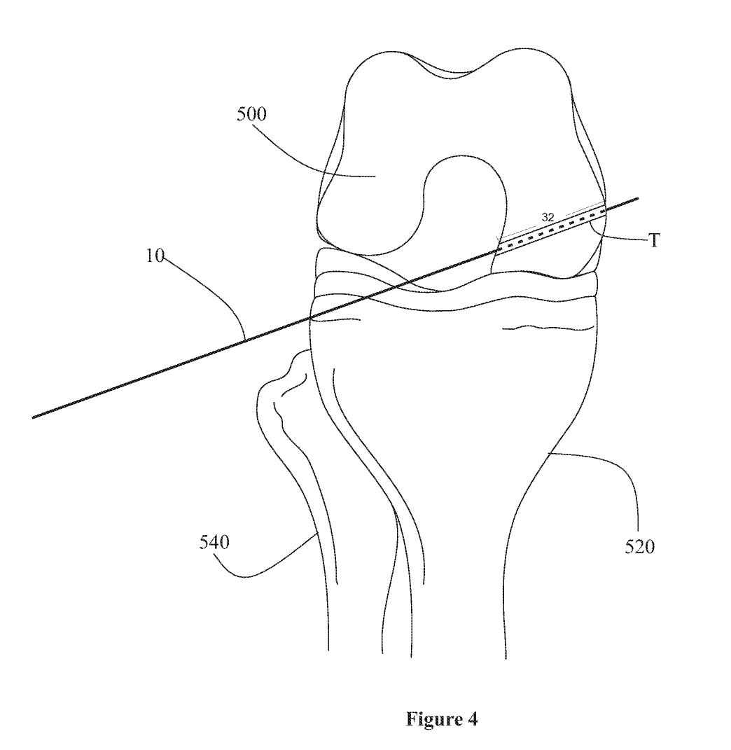

Now referring to FIGS. 1 to 8, in an aspect, a drilling assembly 100 for preparing graft socket for anatomic ligament reconstruction in accordance with the present invention is illustrated. The drilling assembly 100 in the present embodiment is shown to be used for preparing a femoral ACL graft socket tunnel ‘ST’ for anatomic Anterior Cruciate Ligament (herein after ‘the ACL’) reconstruction. Specifically, in all figures, a femur 500, a tibia 520 and a fibula 540 of the knee joint are depicted. It may be obvious to a person skilled in the art to use the drilling assembly 100 for graft socket tunnel construction at other joints as well.

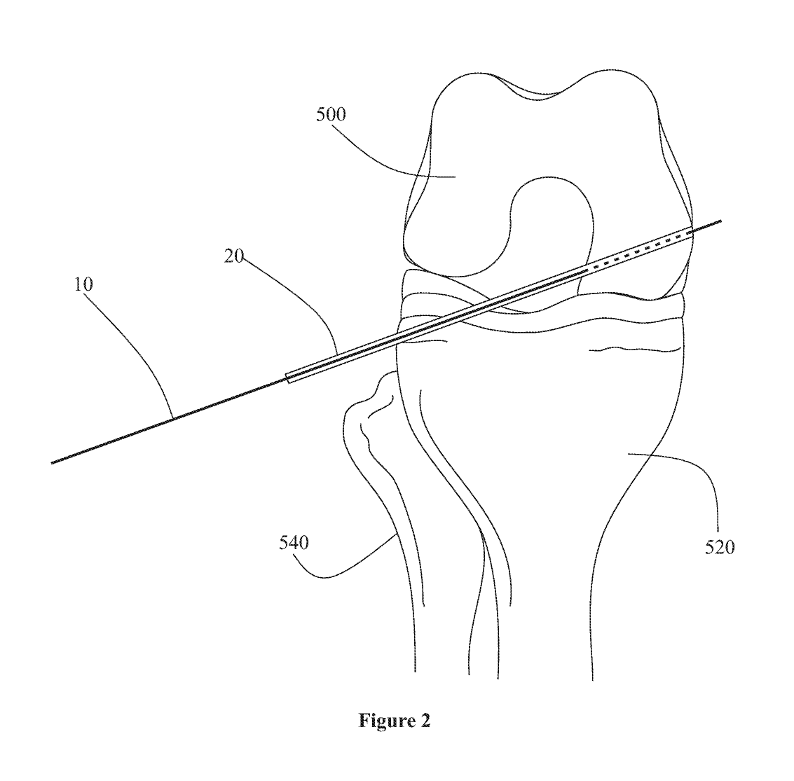

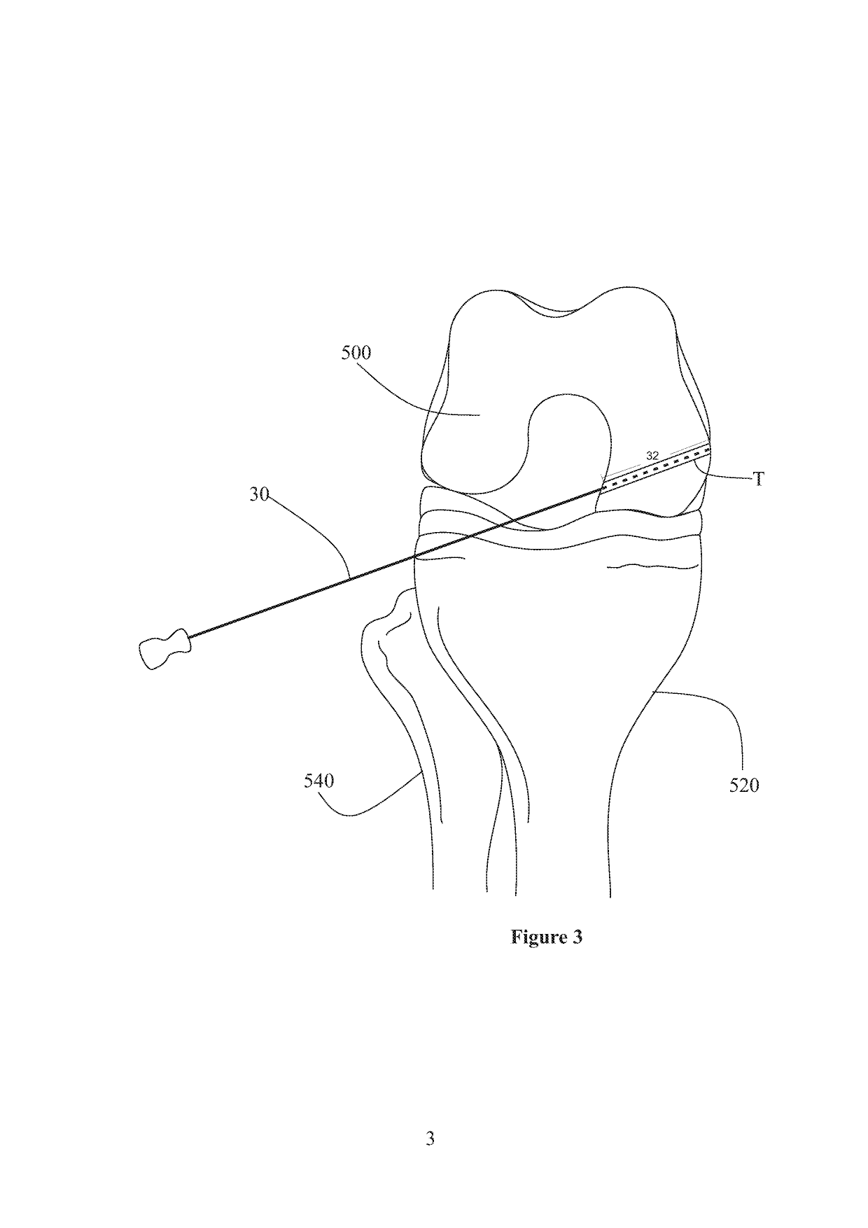

Further, the ACL reconstruction is carried out by passing and drilling a guide wire 10 through a femoral aimer (not shown) with knee flexed at 120 degrees through an Antero Medial (AM) portal at an anatomical ACL femoral insertion site (refer FIG. 1) for determining the direction of the femoral ACL graft socket tunnel ‘ST’. Thereafter, a larger drill 20 of about 4.5 mm diameter is passed over the guide wire 10 to drill a tunnel in a lateral femoral condyle as shown in FIG. 2. The length of the tunnel “T” is measured using a depth gauge 30 as shown in FIG. 3. The surgeon based on the measured tunnel length decides the femoral ACL graft socket length. In the present embodiment, the tunnel length is 32 mm therefore to configure the femoral ACL graft socket tunnel an additional drill of diameter 6-11 mm and about a length of 25 mm has to be drilled.

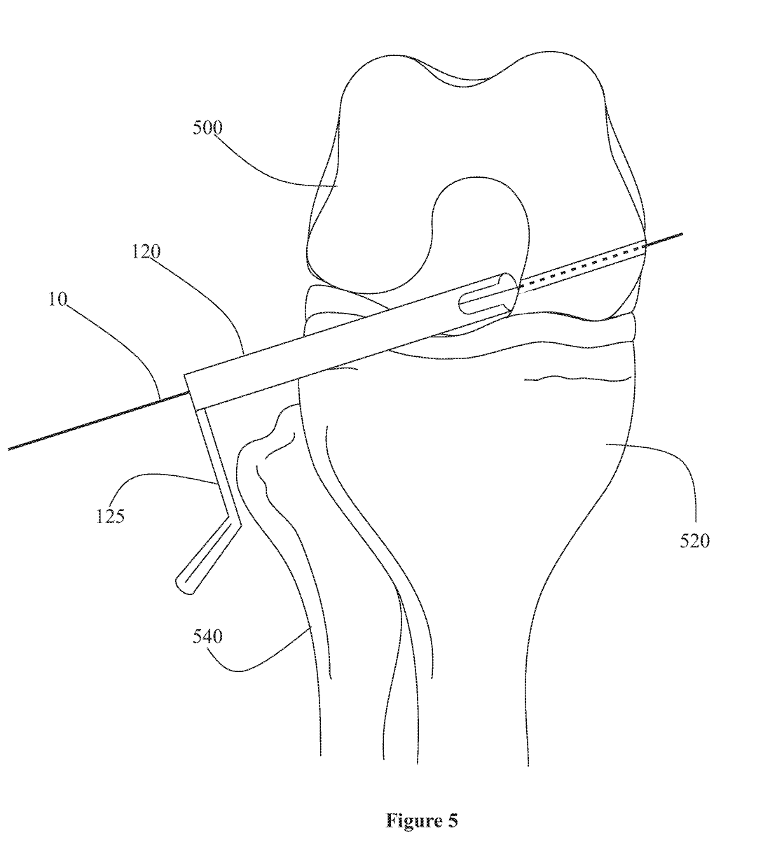

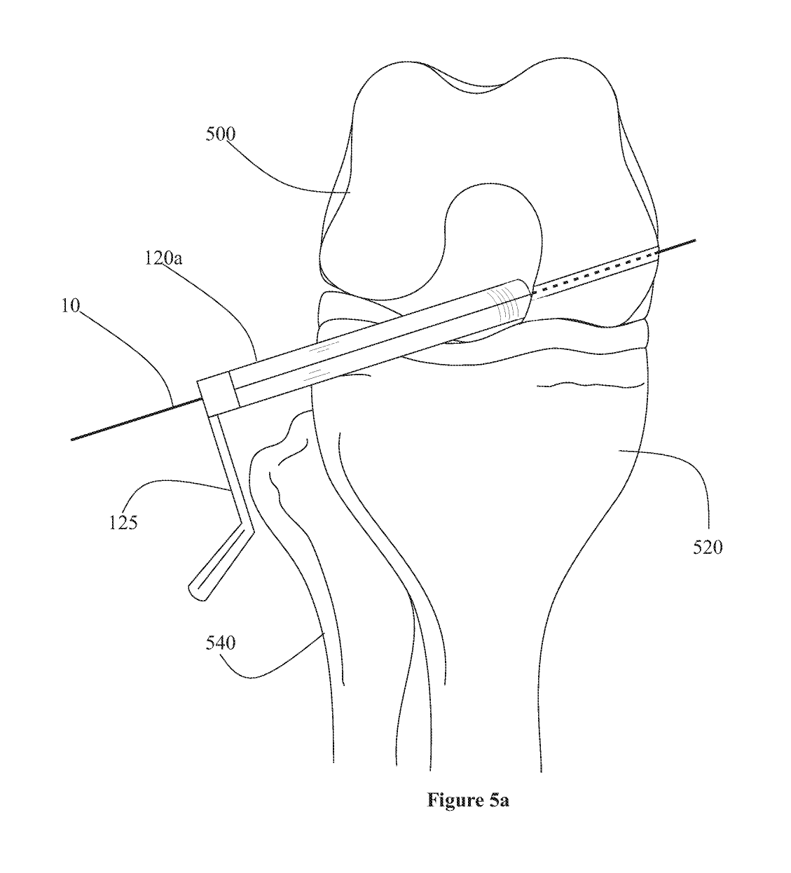

Referring now to FIGS. 5 to 8, the drilling assembly 100 in accordance with the present invention is illustrated. The drilling assembly 100 includes an endoscopic reamer (herein after ‘the reamer’) 110, an outer sleeve 120 and a stopper 130.

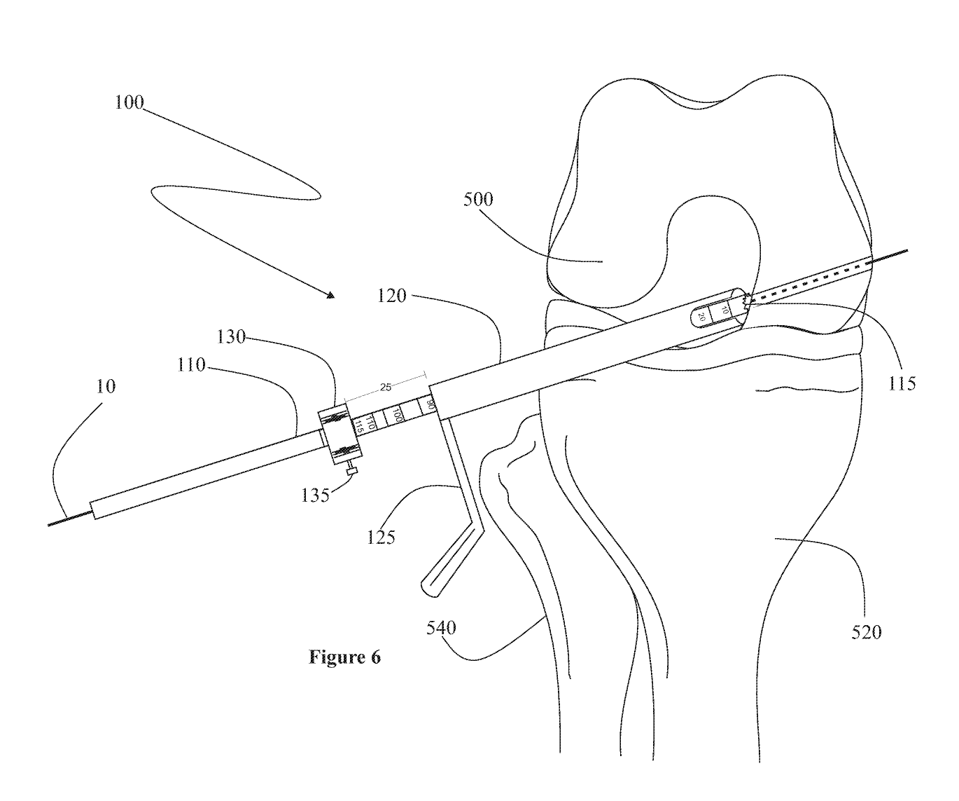

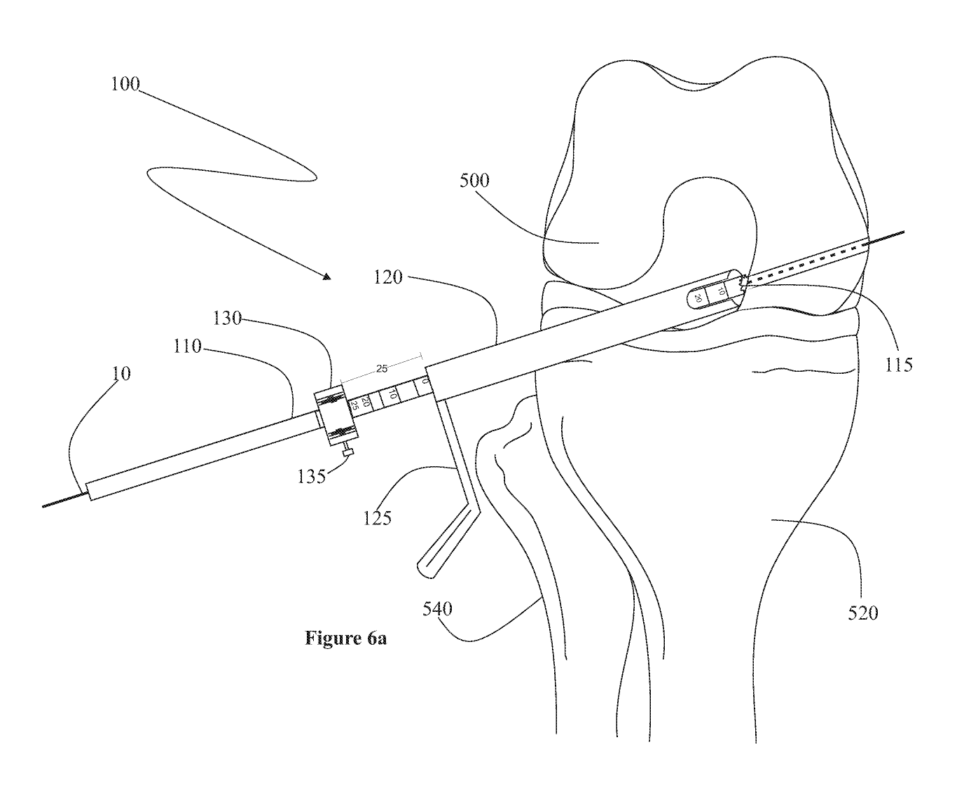

The reamer 110 is a femoral endoscopic drill of a size ranging between 6 mm to 11 mm. The reamer 110 includes at least one measurement marking thereon. For example, an end on which a drilling tool 115 is mounted is marked ‘0’ and scale is extended away from the drilling tool 115 in mm. In another embodiment and as shown in FIG. 6a, the reamer 110 has dual length measurement markings thereon, a first measurement starting from the drilling tool 115 as it starts on a slandered reamer and a second measurement after the length of the outer sleeve 120. For example, the length of the outer sleeve 120 is 90 mm, then the second measurement will read ‘0’ at 90 mm and the measurement marking in mm will be marked along the length away from the drilling tool 115. This reduces calculation requirements.

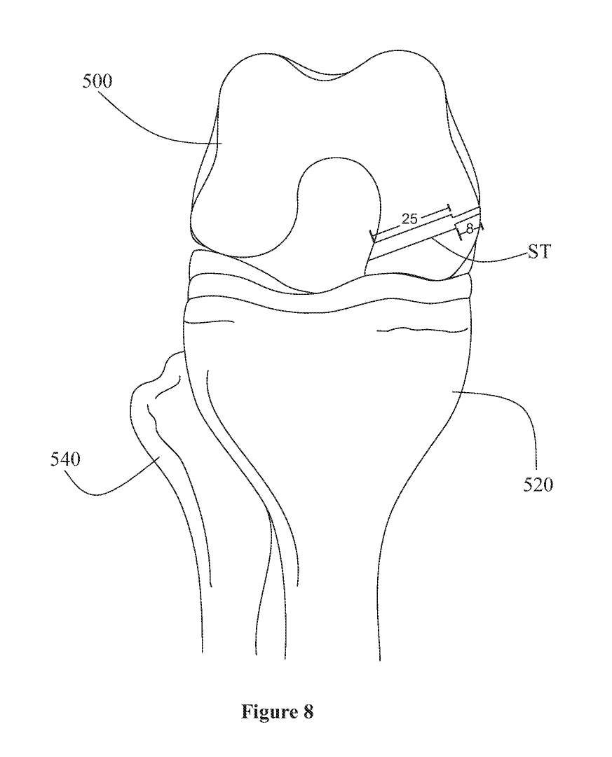

Further, the diameter of the reamer 110 is selected based on the diameter of the anterior cruciate ligament graft to be inserted at the ACL insertion site. The reamer 110 is adapted to slidably pass over the guide wire 10 for drilling a tunnel of predefined length to prepare the femoral ACL graft socket tunnel “ST” in the lateral femoral condyle for the insertion of an anterior cruciate ligament graft therein. Arthroscopic visualization of the length marking starting from the tip on the reamer 110 confirms the depth of the reaming of the femoral ACL graft socket tunnel “ST” as shown in FIG. 8.

Referring again to FIGS. 5 to 8, the outer sleeve 120 is a fixed length outer protective sleeve of slightly wider diameter than the selected endoscopic reamer. In the present embodiment, the outer sleeve 120 has a length of 90 mm and internal diameter between 6.2-11.2 mm. The outer sleeve 120 is adapted to slidably pass over the guide wire 10 and advanced over the guide wire 10 to rest on the medial wall of a lateral femoral condyle as shown in FIG. 5. Narrow external diameter of the outer sleeve 120 corresponding to the size of the reamer 110 makes passage of the outer sleeve 120 easy through the AM portal. Alternatively as shown in FIG. 5a, an outer sleeve 120a is having half circumference cylindrical potion throughout the length allowing easy insertion of the reamer 110 through the AM portal. Further, the outer sleeve 120 has protective and dual check functions. The outer sleeve 120 has a smooth edge/tip to protect and ensures no damage to the cartilage over the medial femoral condyle (MFC) and fibers of Posterior Cruciate Ligament (PCL) during introduction through the AM portal and advancement over the guide wire 10. It also protects the cartilage over the medial femoral condyle (MFC)and fibers of Posterior Cruciate Ligament (PCL) from the sharp ends of the reamer during insertion and advancement of the reamer through the AM portal. The outer sleeve 120 also provides an end point for the stopper 130 during reaming.

Alternatively, the outer sleeve 120 includes a handle 125 facilitating the surgeon in gripping the outer sleeve 120 during reaming process. The handle 125 is configured to provide a firm grip to the surgeon during the entire process of reaming. The outer sleeve 120 with full circumference cylindrical portion also includes a window configured 122 on an anterior end toward the drilling tool 115. The window 122 is configured on the outer sleeve 120 at an end/tip portion thereof rests over the medial wall of the lateral cortex. The window 122 is configured to provide vision of the scale on the reamer 110 to the surgeon as a secondary check of the length of the drilled femoral ACL graft socket tunnel as the markings on the scale, which begins from the tip of the reamer 110, are seen through the window 122. Thus, the window 122 allows the surgeon arthroscopically to confirm the drilling of the femoral ACL graft socket tunnel “ST” up-to the predefined length as seen by the arthroscope. After reaming the tunnel “T” to configure the femoral ACL graft socket tunnel “ST”, the reamer 110 and the outer sleeve 120 are retracted leaving the guide wire 10 in place.

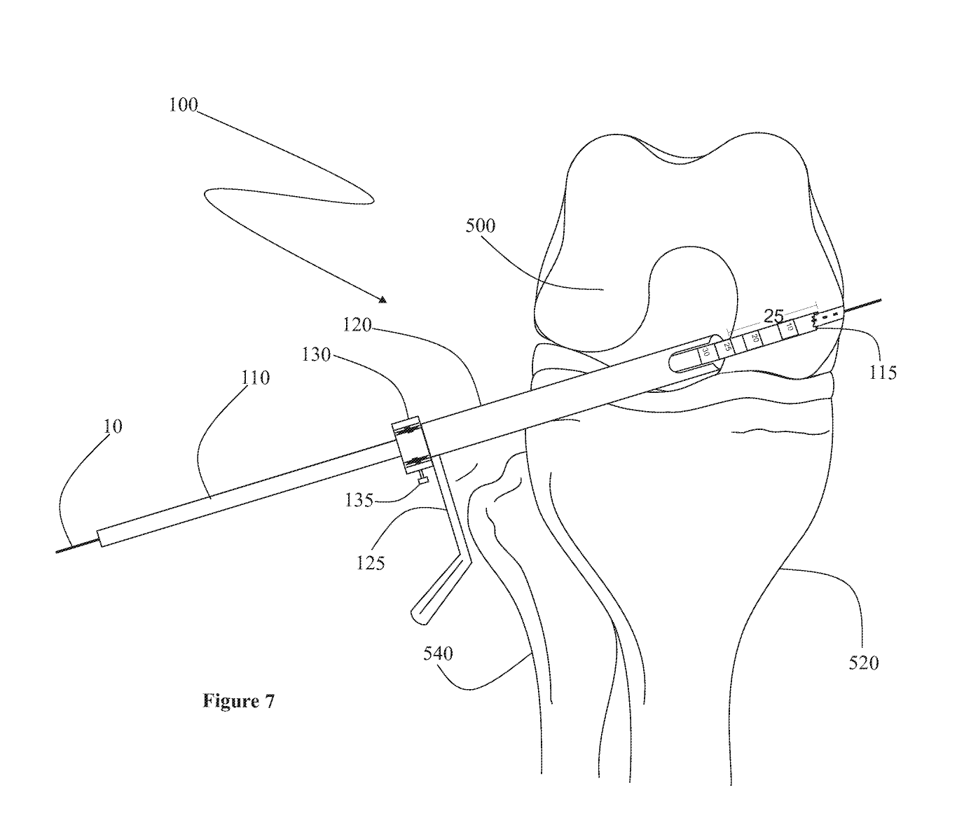

The stopper 130 is adapted to be preloaded and secured at a portion on the reamer 110 enabling the stopping of the travel of the reamer 110 for drilling hole of the predefined length of the femoral ACL graft socket tunnel “ST”. The portion is selected based on the length of the outer sleeve 120 and the length of reaming to configure the femoral ACL graft socket tunnel “ST”. An additional measuring scale on the reamer 110 can be provided starting from 0, allows the surgeon to directly fix the stopper 130 on the desired length without requiring any calculations. The next scale marking depends upon the length of the outer sleeve 120. In an embodiment, the stopper 130 is manually tightened to the reamer 110 at the length marking corresponding to the predefined length of the femoral ACL graft socket tunnel “ST” i.e. the tunnel to be reamed. Specifically, a hole with inner threading is configured on the stopper 130 and a bolt 135 is passed there through as shown in FIGS. 6 and 7. The bolt 135 on tightening secured the stopper 130 with the reamer 110 rigidly. This arrangement provides a positive grip restricting further drilling upon hitting the stopper 130 by the outer sleeve 120.

The reamer 110 along with the stopper 130 is adapted to be attached on a motorized drill for passing over the guide wire 10 and moving inside the outer sleeve 120 until the stopper 130 hits the outer sleeve 120 thereby avoiding over drilling of the femoral ACL graft socket tunnel “ST” beyond the predefined length.

In a preferred embodiment, the stopper 130 is a variable external stopper having a diameter wider than that of the outer sleeve 120 so as to hit the outer sleeve 120 upon reaching predefined length of the reamer 110 corresponding to the predefined length of the femoral ACL graft socket tunnel “ST” to be drilled thereby avoiding over drilling of the femoral ACL graft socket tunnel beyond the predefined length and provides a secondary protective mechanism for manual controlled drilling.

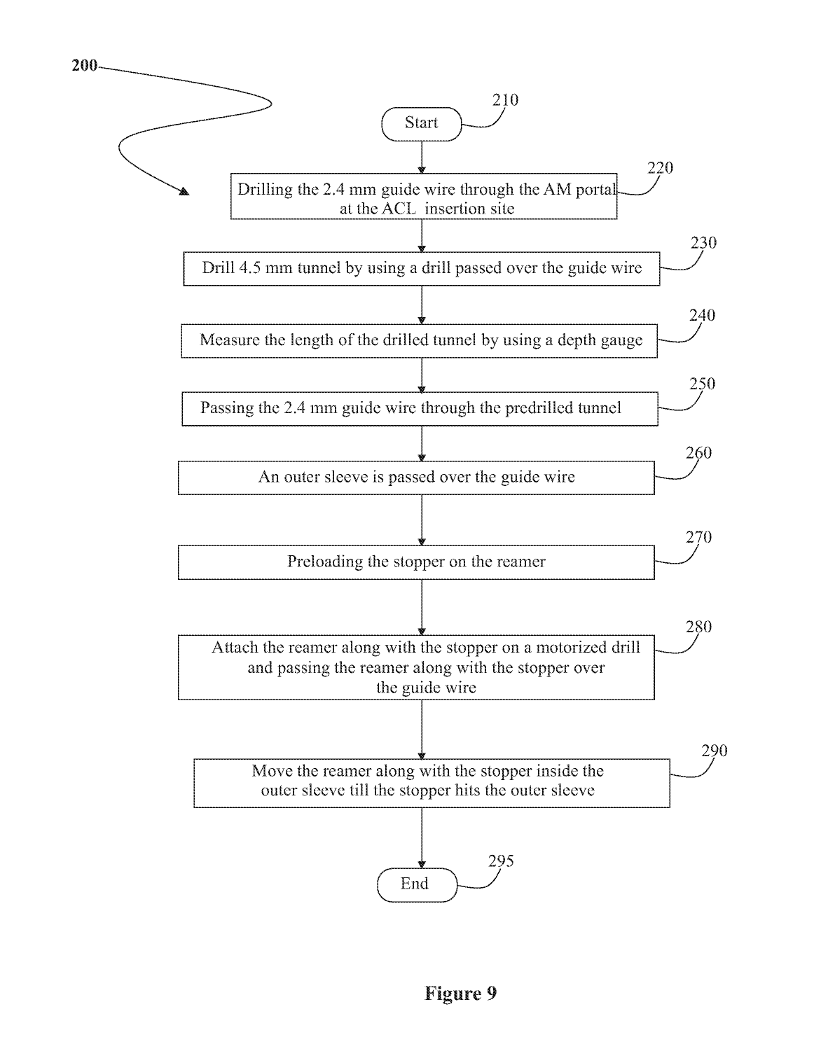

Referring now to FIG. 9, in another aspect, a flow chart of a method 200 for preparing graft socket for ligament reconstruction, specifically, for preparing a femoral ACL graft socket tunnel ‘ST’ for anatomic anterior cruciate ligament ACL reconstruction is illustrated in accordance with the present invention. Specifically, the method 200 is illustrated in conjunction with the drilling assembly 100 and FIGS. 1 to 8 of the present invention for the sake of brevity.

The method starts at step 210.

Initially at step 220, the method 200 involves passing of the guide wire 10 through the AM portal on the knee flexed 120 degrees or more, for determining the direction of the femoral ACL graft socket tunnel Preferably, the guide wire 10 of 2.4 mm is passed and drilled through the AM portal at the anatomic ACL insertion site as shown in FIG. 1.

Thereafter at step 230 the drill 20 of about 4.5 or 5 mm is passed over the guide wire 10 and the femoral tunnel “T” is drilled as shown in FIG. 2.

At step 240, the length of the tunnel “T” is measured in mm using the depth gauge 30.

Thereafter at step 250, the guide wire 10 is again passed through the tunnel “T”.

At step 260, the outer sleeve 120 is passed over the guide wire 10. The outer sleeve 120 with slightly wider diameter than the selected endoscopic reamer 110 is adapted to be slidably passed over the guide wire 10 and advanced over the guide wire 10 to rest on the medial wall of the lateral femoral condyle as shown in FIG. 5.

In a next step 270, the method 200 involves preloading the stopper 130 on the reamer 110, and manually tightening the stopper 130 so as to fit on the reamer 110 on a portion corresponding to the length of the predefined femoral ACL graft socket tunnel to be drilled. In a preferred embodiment, the dual markings on the reamer 110 help the surgeon to easily judge the position where the stopper 130 has to be placed before tightening it.

In a further step 280, the method 200 involves attaching the reamer 110 along with the stopper 130 on a motorized drill and passing the reamer 110 along with the stopper 130 over the guide wire 10 for being introduced inside the outer sleeve 120 as shown in FIG. 6 and is advanced/moved inside the outer sleeve 120 till it reaches the medial wall of the lateral femoral condyle.

Thereafter, at step 290, the reamer 110 along with the stopper 130 is used for drilling the anatomic ACL graft insertion site until the stopper 130 hits the outer sleeve 120 preventing over drilling so as to prepare the predefined length of the femoral ACL graft socket tunnel in the lateral femoral condyle.

Thereafter, the method involves retracting the reamer 110 and the outer sleeve 120 following reaming leaving the guide wire 10 in place and the method 200 ends at step 295.

For confirmation of the intact walls of the femoral ACL graft socket tunnel and intact lateral cortex of the lateral femoral condyle, the surgeon can carry out arthroscopic visualization through the AM portal depicting tunnel-in tunnel appearance.

The remaining steps in the ACL reconstruction process are carried out as routine and hence not described herein detail.

### ADVANTAGES OF THE INVENTION

Avoids Over drilling:

Existing techniques have no protective mechanism to avoid lateral cortex perforation with the endoscopic reamer. Hyperflexion of the knee and bone debris produced during femoral ACL tunnel preparation restricts arthroscopic vision making reaming tricky and operator dependent, prone to manual error or wrong judgment. The drilling assembly of the present invention has the advantage of precise reaming in which the variable position stopper is tightened and fixed to the reamer at the length marking that corresponds to the predefined length of the tunnel to be drilled. The stopper avoids over drilling of the femoral ACL graft socket tunnel and provides a secondary protective mechanism for manual controlled drilling.

### Dual Check Mechanism:

The window in the full circumference cylindrical portion of the outer sleeve or an outer sleeve with half circumference cylindrical portion provides vision for secondary check of the length of the tunnel being drilled. Arthroscopic visualization of the length marking starting from the tip on the reamer confirms the depth of the reaming of the femoral ACL graft socket tunnel This dual check mechanism confirms and provides a dual confirmation of the length of femoral ACL graft socket tunnel drilling.

Protective Function of the sleeve:

The outer sleeve protects the cartilage over the MFC and fibers of the PCL from the sharp ends of the endoscopic reamer during insertion and advancement of the reamer through the AM portal. The outer sleeve also provides an end point for the variable external stopper during reaming. Narrow external diameter of the outer protective sleeve corresponding to the size of the endoscopic reamer makes passage of the sleeve easy through the AM portal. Smooth edges of the outer sleeve ensure no damage to the cartilage of the MFC and fibers of the PCL during insertion and advancement over the guide wire. The handle on the outer sleeve provides a firm grip to the holder during the entire process of reaming.

The foregoing descriptions of specific embodiments of the present invention have been presented for purposes of illustration and description. They are not intended to be exhaustive or to limit the present invention to the precise forms disclosed, and obviously many modifications and variations are possible in light of the above teaching. The embodiments were chosen and described in order to best explain the principles of the present invention and its practical application, to thereby enable others skilled in the art to best utilize the present invention and various embodiments with various modifications as are suited to the particular use contemplated. It is understood that various omission and substitutions of equivalents are contemplated as circumstance may suggest or render expedient, but such are intended to cover the application or implementation without departing from the spirit or scope of the claims of the present invention.

What is claimed is:

1. A drilling assembly for preparing graft socket for ligament reconstruction, the ligament reconstruction being carried out by passing and drilling a guide wire at ligament insertion site for determining the direction of the graft socket tunnel, thereafter passing a larger drill (about 4.5 or 5 mm) of predefined diameter over the guide wire to drill the tunnel in a bone throughout the entire length, measuring the length of the tunnel using a depth gauge, the ligament graft socket tunnel length being determined by the surgeon based on the measured tunnel length, characterised in that the drilling assembly comprising:
an endoscopic reamer with at least one measurement marking thereon adapted to slidably pass over the guide wire for drilling a predefined length marking to prepare the graft socket tunnel in the bone for the insertion of the ligament graft therein;
an outer sleeve with slightly wider diameter than the selected endoscopic reamer adapted to be slidably passed over the guide wire and advanced over the guide wire to rest on the inner cortex of the bone; and
a stopper adapted to be preloaded and secured to the endoscopic reamer at a portion on the endoscopic reamer enabling the stopping of the travel of the endoscopic reamer for drilling hole of a predefined length on the graft socket.

2. The drilling assembly as claimed in claim 1, wherein the endoscopic reamer has dual measurement markings indicated thereon.

3. The drilling assembly as claimed in claim 1, wherein the endoscopic reamer is an endoscopic drill of a size ranging between 6 mm to 11 mm.

4. The drilling assembly as claimed in claim 1, wherein the size of the endoscopic reamer is selected based on the size of the ligament graft to be inserted at the graft socket tunnel.

5. The drilling assembly as claimed in claim 1, wherein the endoscopic reamer along with the stopper is adapted to be attached on a motorized drill for passing over the guide wire and moving inside the outer sleeve which has a slightly wider diameter than the selected endoscopic reamer, drilling the tunnel until the stopper hits the outer sleeve thereby avoiding over drilling of the graft socket tunnel beyond the predefined length.

6. The drilling assembly as claimed in claim 1, wherein the outer sleeve is a fixed length outer protective sleeve that includes a handle configured thereon to provide grip to a surgeon.

7. The drilling assembly as claimed in claim 1, wherein the outer sleeve includes a window configured at the tip to allow a surgeon to arthroscopically confirm the drilling of the graft socket tunnel up-to the predefined length.

8. The drilling assembly as claimed in claim 1, wherein the window is configured at the tip of the outer sleeve with full circumference cylindrical portion for providing visibility for measurement markings on the reamer to the surgeon.

9. The drilling assembly as claimed in claim 1, wherein the outer sleeve has a half circumference cylindrical portion for providing visibility for measurement markings on the reamer and easy insertion through the portal for the surgeon.

10. The drilling assembly as claimed in claim 1, wherein the stopper is a variable external stopper having a diameter wider than that of the outer sleeve so as to hit the outer sleeve upon reaching at least one measurement marking on the endoscopic reamer corresponding to the predefined length of the graft socket tunnel to be drilled thereby avoiding over drilling.

11. The drilling assembly as claimed in claim 1, wherein the stopper has a hole with threading for passing a bolt therefore gripping the stopper rigidly on the reamer.

12. A method for graft socket preparation for anatomic anterior cruciate ligament reconstruction, the method comprising:
calculating and pre-deciding a length of graft socket tunnel for a ligament insertion site to be drilled through an Antero Medial portal in a flexed joint using an endoscopic reamer for preparing a graft socket tunnel in a lateral femoral condyle for the insertion of an anterior cruciate ligament graft therein by a surgeon, the endoscopic reamer having at least one measurement marking thereon, the endoscopic reamer selected based on a size of the ligament graft to be inserted;
preloading a stopper on the endoscopic reamer, and tightening the stopper so as to fit on the endoscopic reamer at least one measurement marking corresponding to the length of the predefined tunnel to be drilled;
attaching the endoscopic reamer along with the stopper on a motorized drill;
passing the endoscopic reamer along with the stopper over a guide wire for being introduced inside an outer sleeve which has slightly wider diameter than the selected endoscopic reamer and advancing till it reaches the medial wall of the lateral femoral condyle and moving therein for drilling the insertion site until the stopper hits the outer sleeve preventing over drilling so as to prepare the predefined length of the tunnel in the lateral femoral condyle;
passing an outer sleeve via a handle thereof over the guide wire and advanced to rest over the medial wall of the lateral femoral condyle to protect the cartilage over the medial femoral condyle and fibers of Posterior Cruciate Ligament during drilling;
arthroscopically confirming the measurement markings over the endoscopic reamer depicting the length of the drilled graft socket tunnel through a window configured at the tip end of the sleeve; and
retracting the endoscopic reamer and the outer sleeve following reaming leaving the guide wire in place.
